(19) United States
(12) Patent Application Publication
(10) Pub. No.: US 2023/0248848 A1
(43) Pub. Date: Aug. 10, 2023

(54) METHODS AND COMPOSITION FOR DYE-BASED ENCODING AND QUANTIFICATION

(71) Applicant: ARIZONA BOARD OF REGENTS ON BEHALF OF THE UNIVERSITY OF ARIZONA, Tucson, AZ (US)

(72) Inventor: Jared Churko, Tucson, AZ (US)

(21) Appl. No.: 18/166,390

(22) Filed: Feb. 8, 2023

Related U.S. Application Data
(60) Provisional application No. 63/307,930, filed on Feb. 8, 2022.

(51) Int. Cl. A61K 49/00

(52) U.S. Cl. CPC A61K 49/0054; A61K 49/0043; A61K 49/0021

(57) ABSTRACT

Fluorescently tagged nucleotides have a wide array of uses; however, methods of quantifying said nucleotides are lacking. The present invention features compositions and methods for quantifying cellular dyes within a sample. Compositions described herein comprise an oligonucleotide barcode conjugated to a cellular dye.

### CROSS-REFERENCES TO RELATED APPLICATIONS

This application is a non-provisional and claims benefit of U.S. Provisional Application No. 63/307,930 filed Feb. 8, 2022, the specification of which is incorporated herein in its entirety by reference.

### FIELD OF THE INVENTION

The present invention features methods and compositions for the quantification of cellular dyes and, thus, cellular components.

### BACKGROUND OF THE INVENTION

Fluorescently tagged nucleotides have a wide array of uses. One of the most widely used applications for fluorescently tagged nucleotides is correlating fluorescence intensity values with gene expression values in real-time PCR. With the advent of more sophisticated genomic sequencing methodologies, further applications using oligonucleotide conjugated dyes can accelerate research.

Various methods to capture single cells for downstream single-cell RNA-seq exist, such as microfluidic based capture (e.g., endogenous Fluidigm C1), droplet-based capture (e.g., 10× Genomics or Drop-seq), and microwell-based capture (e.g., Seq-well or BD Rhapsody). Once individual cells are isolated, transcripts within the cell are typically “barcoded” with a unique oligonucleotide sequence. Then, all cells are lysed sequenced using high-throughput sequencing methodology, and gene expression of a single cell can be quantified by counting the transcripts containing the same barcode (i.e., cell demultiplexing). Similarly, another barcode can be added to all cells within that sample, and after sequencing, samples can be demultiplexed (e.g., comparing control versus treated samples or wild-type versus mutant samples). Current methods to add these sample specific barcode tags typically are at the library preparation stage (PCR or ligase-based); however, this barcode can be added during the live-cell stage by adding antibodies conjugated to oligonucleotides against cell surface proteins. This is currently sold as a commercial Multiplexing Kit (BD, Cat. No. 633781), where up to 12 samples can be run on a single experimental chip. However, these antibody-based kits are limited by the surface antigen being expressed within the targeted sample. Thus, different kits are needed for human versus mouse samples, nuclear versus whole-cell samples, and heterogeneity cell surface marker expression samples (not all cells may express the same surface antigen).

### BRIEF SUMMARY OF THE INVENTION

It is an objective of the present invention to provide compositions, methods, and kits that allow for quantifying cellular dyes in a plurality of samples, as specified in the independent claims. Embodiments of the invention are given in the dependent claims. Embodiments of the present invention can be freely combined with each other if they are not mutually exclusive.

Cellular dyes are often used to label specific organelles or indicate the properties or presence of biological components. For example, MitoTracker labels mitochondria, and dyes targeting the mitochondria can be used to evaluate which cells have a high metabolic/energetic need. In addition, routine dyes such as Hematoxylin and eosin (H&E) stains are commonly used to label acidic and basic biological components such as nucleotides which label for eosin, and proteins which label for Hematoxylin. Thus, the present invention features cellular dyes conjugated to oligonucleotide barcodes. By adding barcodes attached to these cellular dyes, cells enriched for a specific dye would result in more barcodes within the cell. After high-throughput sequencing, quantifying the number of reads containing the barcode can quantify the amount of dye within each cell. Subsequently, this data can be used to infer or act as a surrogate to quantify what the dye is specific for. Furthermore, multiple dyes, each tagged with a unique barcode, can be used to multiplex numerous cellular properties within a single run. Barcoded dyes can also be used to encode spatial information (barcodes are keyed to specific X/Y/Z coordinates), where maps of cellular and biological content can represent particular regions of the cell or tissue section.

The present invention features the use of cellular dyes conjugated to oligonucleotide barcodes to resolve many issues related to antibody-based labeling. As described herein, dye barcodes can be used to quantify dye-based properties (e.g., lipid-based dyes can quantify lipids within each cell along with the single-cell RNA-seq profile).

In some embodiments, the present invention features a composition comprising an oligonucleotide barcode conjugated to a cellular dye, wherein the barcode comprises a plurality of nucleotides. In other embodiments, the present invention features a composition for quantifying endogenous cellular components comprising an oligonucleotide barcode conjugated to a cellular dye, wherein the oligonucleotide barcode comprises a plurality of nucleotides; and wherein the cellular dye binds to a cellular component.

In some embodiments, the present invention features a method of quantifying cellular components (e.g., endogenous cellular components) in one or more samples. The method may comprise a) labeling at least one cellular component (e.g., endogenous cellular component) in the one or more samples with at least one composition comprising oligonucleotide barcode conjugated to a cellular dye, wherein the oligonucleotide barcode comprises a plurality of nucleotides, and wherein the cellular dye binds to the cellular component, and b) quantifying endogenous cellular components using the oligonucleotide barcode

In some embodiments, the present invention features a kit for quantifying endogenous cellular components in one or more samples. The kit may comprise at least one composition comprising an oligonucleotide barcode conjugated to a cellular dye, wherein the oligonucleotide barcode comprises a plurality of nucleotides, and wherein the cellular dye binds to the cellular component. The kit may further comprise at least one primer pair specific to a portion of the oligonucleotide barcode.

One of the unique and inventive technical features of the present invention is the use of cellular dyes conjugated to oligonucleotide barcodes. Without wishing to limit the invention to any theory or mechanism, it is believed that the technical feature of the present invention advantageously provides for a reduction in experimental cost and decreased noise. Additionally, dyes are cheaper, more stable, have less batch-to-batch variation, and can be used to target cell-specific properties (e.g., attaching barcodes to mitochondrial dyes can label cardiomyocytes high populations). Furthermore, the present invention would allow for new data types to be collected as absolute quantification of cellular content is not limited by microscope optics. None of the presently known prior references or work has the unique, inventive technical feature of the present invention.

Furthermore, the prior references teach away from the present invention. For example, antibodies can only label epitopes of proteins. Additionally, prior references are limited by microscope optics to quantify cellular content. However, the present inventions allow for new data types to be collected and new methods for quantifying cellular content.

Additionally, the use of lipophilic moiety (e.g., cholesterol) inhibits the ability of prior references to quantify endogenous cellular components. However, the compositions, methods, and kits described herein allow for the quantification of endogenous cellular components.

Any feature or combination of features described herein is included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows an experimental strategy for creating the probes described herein. Step 1: amine-reactive crosslinker chemistry will be used to crosslink oligonucleotides to a viability dye (e.g., a cellular dye; e.g., Calcein AM). In some embodiments, a reactive group on the dye or nucleotide is functionalized and crosslinked. Step 2: Two separate samples will be treated with two unique oligonucleotide tag conjugate dyes. Step 3: Cells from both samples will be pooled and captured on the BD Rhapsody Single-Cell Analysis System. Single-cell libraries will be made using commercially available kits (BD Whole Transcriptome Analysis (WTA) Amplification Kit). Libraries will be sequenced on a NovaSeq 6000. Fastq files will be demultiplexed using a pipeline, known to one of the ordinary skill in the art, with tag1/tag2 sequences. Clustering and transcriptome analysis will be used to validate this approach.

### DETAILED DESCRIPTION OF THE INVENTION

Before the present compounds, compositions, and/or methods are disclosed and described, it is to be understood that this invention is not limited to specific synthetic methods or to particular compositions, as such may, of course, vary. It is also to be understood that the terminology used herein is for the purpose of describing particular embodiments only and is not intended to be limiting.

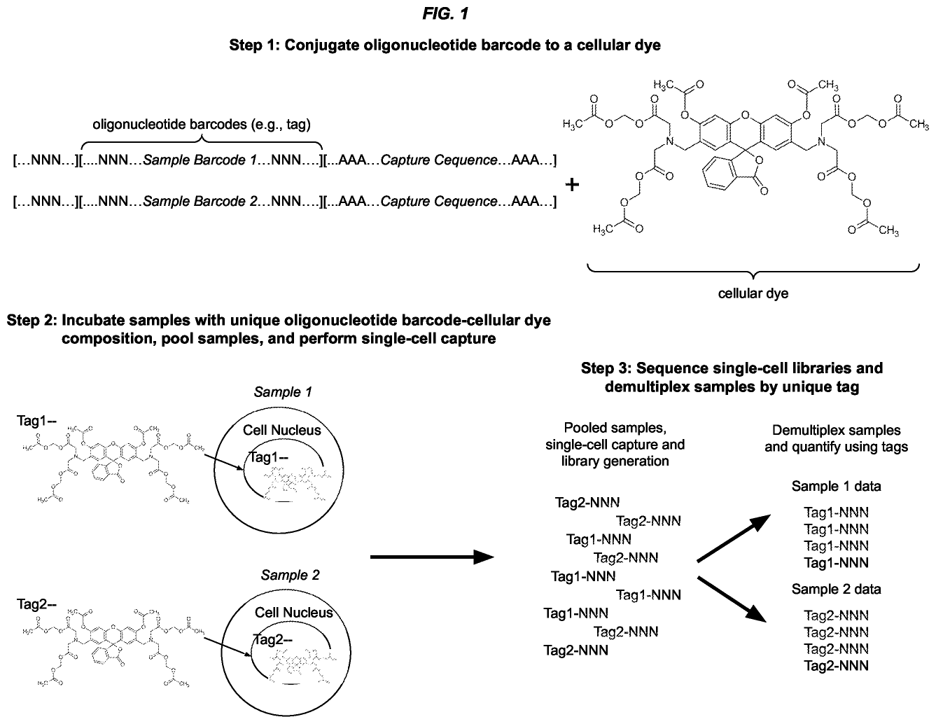

Referring now to FIG. 1, the present invention features compositions, methods, and kits for quantifying a cellular dye and, thus, cellular components (e.g., endogenous cellular components).

As used herein, a “cellular dye” or a “cellular stain” may be used interchangeably and refer to an artificial coloration that is useful in the identification of specific areas within the biological material. Cellular dyes and/or cellular stains may be used to differentiate one or more cellular components or cellular structures from another or to differentiate one or more cellular entities from another in a specimen. As used herein, dyes and/or stains may further be used to differentiate and examine cell populations within tissues, to mark cells, or to flag proteins. In certain embodiments, a dye may refer to a compound comprising a chromophore and auxochrome groups attached to one or more benzene rings, wherein the compound's color is due to the chromophore and its dyeing affinities to the auxochrome.

The present invention features a composition for quantifying cellular components (e.g., endogenous cellular components) comprising an oligonucleotide barcode conjugated to a cellular dye. The oligonucleotide barcode may comprise a plurality of nucleotides. Alternatively, the oligonucleotide barcode may comprise a plurality of nucleotides and a unique molecular identifier (UMI) sequence. In some embodiments, the oligonucleotide barcode may comprise a plurality of nucleotides, a unique molecular identifier (UMI) sequence, and a capture sequence. The cellular dye may bind to a cellular component.

In some embodiments, the oligonucleotide barcode is conjugated to a cellular dye at an N-terminal, e.g., using amine reactive chemistry. In some embodiments, the oligonucleotide barcode is conjugated to a cellular dye at a C-terminal. In some embodiments, the oligonucleotide barcode is conjugated directly to the cellular dye. In other embodiments, the oligonucleotide barcode is conjugated indirectly to the cellular dye, e.g., via a linker. In other embodiments, the oligonucleotide barcode is conjugated to the cellular dye via a linker. A linker may be used to allow for space between the oligonucleotide barcode and the dye. In some embodiments, the linker comprises a known sequence (e.g., a nucleotide sequence) that may be used to amplify the downstream barcode region.

As used herein, a “cellular component” generally refers to complex biomolecules and structures of which cells, and thus living organisms, are composed. The cellular components may comprise DNA, RNA, organelles, proteins, or any combination thereof, from a cell. In some embodiments, the cellular component comprises an endogenous cellular component. In some embodiments, the cellular component comprises an intracellular component (e.g., components within a cell), an extracellular component (e.g., components outside a cell), or a combination thereof (e.g., components both inside and outside of a cell; e.g., transmembrane proteins).

As used herein, an “oligonucleotide barcode” or “barcode” may be used interchangeably and generally refers to a label or identifier that conveys or is capable of conveying information. For example, an oligonucleotide barcode may comprise a plurality of nucleotides that uniquely identifies a molecule (e.g., a cellular dye) to which it is conjugated to. In other embodiments, the oligonucleotide barcode may comprise a plurality of nucleotides that uniquely identify a cell or sample the oligonucleotide barcode is added to. A barcode may be unique. Barcodes can allow for the identification and/or quantification of individual sequencing reads.

In some embodiments, the oligonucleotide barcodes may further comprise a unique molecular identifier (UMI) sequence, e.g., short nucleotide sequences used to uniquely tag the oligonucleotide barcodes described herein. In some embodiments, UMIs may comprise a plurality of nucleotides, e.g., a random unique nucleotide sequence. The UMIs may serve to provide a unique identifier of the starting oligonucleotide barcode that was captured in order to allow quantification of the number of original molecules present (e.g., oligonucleotide barcodes). Additionally, UMIs may help to eliminate amplification bias.

The compositions described herein may comprise a single cellular dye conjugated to a plurality of oligonucleotide barcodes (e.g., a singular dye conjugated to different barcodes). Alternatively, the composition described herein may comprise a plurality of dyes conjugated to a plurality of oligonucleotide barcodes (e.g., multiple dyes conjugated to different barcodes). In the aforementioned embodiments, i.e., a plurality of dyes conjugated to a plurality of oligonucleotide barcodes, each of the plurality of dyes may comprise a unique oligonucleotide barcode, such that each of the plurality of dyes may be quantified. For example, multiple dyes could be used in a single sample where each dye is distinguished by its unique oligonucleotide barcode.

One of the ordinary skill in the art would understand that an oligonucleotide barcode refers to short sequences used to uniquely tag each dye, sample, cell, and/or molecule. Additionally, one of the ordinary skill in the art would be able to choose sequences for oligonucleotide barcodes using a plethora of tools currently available. In some embodiments, the oligonucleotide barcodes described herein are predetermined. The oligonucleotide barcodes may be predetermined before the oligonucleotide barcodes are conjugated to a cellular dye.

In some embodiments, the oligonucleotide barcodes described herein may comprise about 75 nucleotides (nts) to 150 nts, or about 75 nts to 125 nts, or about 75 nts to 100 nts, or about 100 nts to 150 nts, or about 100 nts to 125 nts, or about 125 nts to 150 nts. Oligonucleotide barcodes that are longer may allow for a more unique nucleotide sequence.

In some embodiments, the oligonucleotide barcode comprises a cellular component barcode comprising a plurality of nucleotides. In some embodiments, the oligonucleotide barcode comprises a cellular component barcode comprising a plurality of nucleotides and a unique molecular identifier (UMI) sequence. In some embodiments, the oligonucleotide barcode comprises a cellular component barcode comprising a plurality of nucleotides and a capture sequence. The plurality of nucleotides in the cellular component barcode is unique with respect to a cellular component (e.g., the cellular component bound by the dye). As used herein, a “cellular component barcode” refers to a plurality of nucleotides used to determine what cellular component (e.g., an endogenous cellular component) the cellular dye was bound to.

In some embodiments, the oligonucleotide barcode comprises a sample barcode comprising a plurality of nucleotides. In some embodiments, the oligonucleotide barcode comprises a sample barcode comprising a plurality of nucleotides and a unique molecular identifier (UMI) sequence. In some embodiments, the oligonucleotide barcode comprises a sample barcode comprising a plurality of nucleotides and a capture sequence. The plurality of nucleotides in the sample barcode is unique with respect to a sample. As used herein, a “sample barcode” refers to a plurality of nucleotides used to determine what sample the dye was added to.

In some embodiments, the oligonucleotide barcode comprises a single-cell barcode comprising a plurality of nucleotides. In some embodiments, the oligonucleotide barcode comprises a single-cell barcode comprising a plurality of nucleotides and a unique molecular identifier (UMI) sequence. In some embodiments, the oligonucleotide barcode comprises a single-cell barcode comprising a plurality of nucleotides and a capture sequence. The plurality of nucleotides in the single-cell barcode is unique with respect to a single cell (e.g., a single cell within a sample). As used herein, a “single-cell barcode” refers to a plurality of nucleotides used to determine what cell within a particular sample the cellular dye was added to.

In some embodiments, the oligonucleotide barcode comprises a spatial barcode comprising a plurality of nucleotides. In some embodiments, the oligonucleotide barcode comprises a spatial barcode comprising a plurality of nucleotides and a unique molecular identifier (UMI) sequence. In some embodiments, the oligonucleotide barcode comprises a spatial barcode comprising a plurality of nucleotides and a capture sequence. The plurality of nucleotides in the spatial barcode is unique with respect to a particular location in a sample (e.g., a particular x, y, and/or z coordinate in a sample). As used herein, a “spatial barcode” refers to a plurality of nucleotides used to determine where in a sample the dye was added.

In some embodiments, the capture sequence comprises an oligo dT. In other embodiments, the capture sequence comprises a random multimer. For example, if capturing mRNA, then an oligo dT capture sequence may be used; alternatively, if capturing a specific gene and/or RNA, a random multimer of the gene and/or RNA itself may be used. As used herein, a “capture sequence” refers to a plurality of nucleotides used to enrich genomic regions of interest and may be used for library preparation and sequencing.

In some embodiments, the capture sequence is used for a reverse transcription reaction.

In some embodiments, the compositions described herein may comprise any cellular dye known to those skilled in the art. In some embodiments, the cellular dyes described herein may label cellular organelles or structures, including but not limited to lysosomes, cytoskeleton, nucleus, mitochondria, exosomes, cytoplasm, lipid droplets, vesicles, membrane and cell surface, RNA granules. In some embodiments, the cellular dyes described herein may label components specific for a diseased cell, including but not limited to amyloid plaques (e.g., beta-amyloid plaques in Alzheimer's Disease with Congo Red and Thioflavin S stain) or fibrosis (e.g., collagen with aniline blue or picrosirius red). In further embodiments, the cellular dyes described herein may infer cell types. For example, ratios between cytoplasmic and nuclei dyes can tell you how much nuclear content is within a cell. Specifically, granulocytes (e.g., neutrophils, eosinophils, and basophils) have granules that can be detected using a dye (e.g., a mitochondrial), and cardiomyocytes are multinucleated and can be detected using a nuclei dye.

Non-limiting examples of cellular dyes that may be used in the compositions and methods described herein include but are not limited to Calcein AM, DAPI, Hoechst, TMRE, DASPEI, thioflavin-S, Congo red, fura-2, or lysosomotropic dyes. Other dyes may be used in accordance with the composition and methods described herein.

In some embodiments, the cellular dyes described herein may comprise membrane-permeable dyes (e.g., lipophilic dyes). In some embodiments, the cellular dyes described herein may comprise small dyes, e.g., dyes less than 1 kD, which can pass through gap junctions in a cell. In some embodiments, a cell membrane may be permeabilized (e.g., using Triton X or methanol/acetone) to allow for a cellular dye (e.g., dyes larger than 1 kD) to pass through the cell membrane.

In some embodiments, cellular dyes that indicate a specific state of a cell (e.g., fura-2) may be used in accordance with the compositions described herein. In some embodiments, the cellular dye may respond to specific metabolites and/or ions. The ability of a cellular dye to respond to specific metabolites and/or ions may allow for the quantification of the amount of ions and/or the functional state that a cell is in. For example, a cellular dye (e.g., fura-2) may change based on the presence or absence of calcium, such that the cellular dye (e.g., fura-2) changes its configuration to an open state when bound by calcium, which could either sequester that dye within the cell or expose the barcode region (e.g., the oligonucleotide barcode) to be read.

In some embodiments, the cellular dye binds to a cellular component. In some embodiments, the cellular dye binds to an endogenous cellular component. In some embodiments, the cellular dye binds to an intracellular cellular component, an extracellular cellular component, or a combination thereof. In some embodiments, the cellular dye binds to an endogenous intracellular cellular component, an endogenous extracellular cellular component, or a combination thereof.

In some embodiments, the compositions described herein may be used for multiplexing and demultiplexing a plurality of samples. In some embodiments, the compositions described herein are used for high-throughput sequencing (e.g., single-cell high-throughput sequencing).

The present invention may also feature a method of quantifying endogenous cellular components in one or more samples. The method may comprise a) labeling at least one endogenous cellular component in the one or more samples with at least one composition comprising an oligonucleotide barcode conjugated to a cellular dye configured to bind to the endogenous cellular component and b) quantifying the endogenous cellular component using the oligonucleotide barcode (e.g., dyes which preferentially bind to the mitochondria can be used to quantify the levels of mitochondria in a cell by using the oligonucleotide barcode). In some embodiments, the oligonucleotide barcode comprises a plurality of nucleotides and a capture sequence.

In some embodiments, quantifying the endogenous cellular component comprises sequencing the oligonucleotide barcode (e.g., an oligonucleotide barcode comprising a UMI) to determine how many copies of the oligonucleotide barcode (e.g., an oligonucleotide barcode comprising a UMI) were in a sample.

In some embodiments, the method may comprise labeling one or more endogenous cellular components with one or more compositions described herein. For example, if two endogenous cellular components are being labeled, two different compositions described herein may be used (e.g., a composition comprising a cellular component barcode conjugated to a nuclear dye and a composition comprising a cellular component barcode conjugated to a mitochondrial dye).

In some embodiments, the method may further comprise labeling the one or more samples with one or more compositions described herein. The sample labeling barcode may comprise a plurality of nucleotides that are unique with respect to the one or more samples. The sample labeling barcode may comprise a plurality of nucleotides that are unique with respect to the one or more samples and a capture sequence.

In some embodiments, the method may further comprise labeling one or more cells in a sample with one or more compositions described herein. The single-cell barcode may comprise a plurality of nucleotides that are unique with respect to a single cell in a sample. The single-cell barcode may comprise a plurality of nucleotides that are unique with respect to a single cell in a sample and a capture sequence.

In some embodiments, the present invention features a method for quantifying a cellular dye in a sample. The method may comprise a) labeling a sample with a composition comprising an oligonucleotide barcode conjugated to a cellular dye and b) quantifying the cellular dye using the oligonucleotide barcode. In some embodiments, the oligonucleotide barcode comprises a plurality of nucleotides.

In some embodiments, the present invention features a method for quantifying cellular dyes. The method may comprise a) labeling a sample with two or more compositions comprising an oligonucleotide barcode conjugated to a cellular dye and b) quantifying the cellular dye using the oligonucleotide barcode. In some embodiments, each of the two or more compositions comprises a unique cellular dye and oligonucleotide barcode.

In some embodiments, the present invention features a method of quantifying cellular dyes in two or more samples. The method may comprise a) labeling two or more samples with two or more compositions comprising an oligonucleotide barcode conjugated to a cellular dye and b) quantifying the cellular dye using the oligonucleotide barcode. In some embodiments, the oligonucleotide barcode of each composition comprises a sample barcode that is unique to each of the samples.

In some embodiments, the methods described herein further comprise pooling the one or more samples. In some embodiments, the methods described herein further comprise pooling the two or more samples.

In some embodiments, an oligonucleotide barcode may comprise identical sample labeling sequences and the same single-cell labeling sequences.

In some embodiments, the methods described herein may be used to multiplex or demultiplex samples.

The present invention may further comprise a kit for quantifying endogenous cellular components in one or more samples. The kit may comprise at least one composition comprising an oligonucleotide barcode conjugated to a cellular dye, wherein the oligonucleotide barcode comprises a plurality of nucleotides and wherein the cellular dye binds to the cellular component. The kit may further comprise at least one primer pair specific to a portion of the oligonucleotide barcode. In some embodiments, one of the primers from the primer pair is specific to a region upstream of the oligonucleotide barcode. The primers may be used to amplify the oligonucleotide barcode before sending it for sequencing. In some embodiments, the at least one primer pair amplifies the oligonucleotide barcode.

In some embodiments, the oligonucleotide barcode comprises a cellular component barcode, a sample barcode, a single-cell barcode, or a combination thereof. In some embodiments, the cellular component barcode comprises a plurality of nucleotides that are unique with respect to the cellular component bound by the cellular dye. In some embodiments, the sample barcode comprises a plurality of nucleotides that are unique with respect to the one or more samples. In some embodiments, the single-cell barcode comprises a plurality of nucleotides that are unique with respect to a single cell.

### EXAMPLE

The following is a non-limiting example of the present invention. It is to be understood that said example is not intended to limit the present invention in any way. Equivalents or substitutes are within the scope of the present invention.

### Example 1: Proof of Principle

To demonstrate proof of principle in this approach, an oligonucleotide barcode will be conjugated with a cell viability dye (e.g., Calcein AM) using amine-reactive crosslinker chemistry (FIG. 1) in separate reactions. To evaluate two samples (i.e., a human iPSCs sample and a primary mouse cell sample), two different oligonucleotide barcodes will be used, each comprising a unique sample labeling sequence (e.g., oligonucleotide barcode tag1 and oligonucleotide barcode tag2). Cells (e.g., human iPSCs) from sample 1 will be incubated with oligonucleotide barcode tag1 conjugated dye, and cells (e.g., primary mouse cells) from sample 2 will be incubated with oligonucleotide barcode tag2 conjugated dye. First, cells from sample 1 and sample 2 will be pooled and run on a BD® Rhapsody Single-Cell Analysis System. Next, single-cell RNA-seq libraries will be generated as part of the WTA kit (BD), sequenced on an Illumina Novaseq 6000, and run using a demultiplexing pipeline. Accurate sample demultiplexing will validate this approach as a viable demultiplexing and quantification method for single-cell genomic applications.

### Example 2: Labeling and Quantifying Amyloid-Beta (Aβ) Plaques in Brain Tissue Samples

A research team wants to quantify AR plaques in three post-mortem brain tissue samples. Two samples are from patients diagnosed with Alzheimer's Disease, and the other tissue sample is from a healthy donor. The researchers purchase a kit comprising two compositions as described herein. One composition comprises a thioflavin-S dye conjugated to a cellular component barcode comprising a plurality of nucleotides (e.g., a determined plurality of nucleotides), and the other comprises a thioflavin-S dye conjugated to a sample barcode comprising a plurality of nucleotides that are unique with respect to a sample. The compositions are then introduced to each of the three samples. The samples are then lysed and then pooled together. Next, the researchers sequence and analyze the oligonucleotide barcodes. After reviewing the sequencing data, the researchers determined no AR plaques were in the healthy donor. The diseased tissue samples have AR plaques; however, one sample has significantly more AR plaques. The researchers continue their experiments to determine the cause of the increase.

### Example 3: Labeling and Quantifying Mitochondria in a Sample

A student orders a kit comprising a composition described herein. For example, the kit comprises a mitochondrial dye (e.g., DASPEI) conjugated to an oligonucleotide barcode (e.g., a cellular component barcode comprising a plurality of nucleotides that are unique with respect to the cellular component (e.g., mitochondria) bound by the cellular dye (e.g., DASPEI) and a UMI sequence). The student adds the composition to each cell sample. Next, the samples are then lysed and pooled together. The student uses the primers in the kit to amplify the region upstream of the oligonucleotide barcode and sends the samples for sequencing. After a week, the student gets her results and determines that one sample has a higher amount of mitochondria as compared to the other samples.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only, and the claims are not limited by the dimensions of the figures. In some embodiments, descriptions of the inventions described herein using the phrase “comprising” includes embodiments that could be described as “consisting essentially of” or “consisting of,” and as such, the written description requirement for claiming one or more embodiments of the present invention using the phrase “consisting essentially of” or “consisting of” is met.

What is claimed is:

1. A composition for quantifying endogenous cellular components comprising an oligonucleotide barcode conjugated to a cellular dye, wherein the oligonucleotide barcode comprises a plurality of nucleotides; and wherein the cellular dye binds to a cellular component.

2. The composition of claim 1, wherein the oligonucleotide barcode comprises a cellular component barcode comprising a plurality of nucleotides, wherein the plurality of nucleotides is unique with respect to the cellular component bound by the cellular dye.

3. The composition of claim 1, wherein the oligonucleotide barcode comprises a sample barcode comprising a plurality of nucleotides, wherein the plurality of nucleotides is unique with respect to a sample.

4. The composition of claim 1, wherein the oligonucleotide barcode comprises a single-cell barcode comprising a plurality of nucleotides; wherein the plurality of nucleotides is unique with respect to a single-cell.

5. The composition of claim 1, wherein the oligonucleotide barcode further comprises a capture sequence.

6. The composition of claim 5, wherein the capture sequence comprises an oligo dT, or a random multimer.

7. The composition of claim 6, wherein the capture sequence is used for a reverse transcription reaction.

8. The composition of claim 1, wherein the cellular dye comprises a Calcein AM dye or Hoechst.

9. The composition of claim 1, wherein the oligonucleotide barcode further comprises a unique molecular identifier (UMI) sequence.

10. The composition of claim 1, wherein the composition is used for multiplexing and demultiplexing a plurality of samples.

11. The composition of claim 1, wherein the composition is used for high-throughput sequencing.

12. The composition of claim 11, wherein the composition is used for single-cell high-throughput sequencing.

13. A method of quantifying endogenous cellular components in one or more samples; the method comprising:
a) labeling at least one endogenous cellular component in the one or more samples with at least one composition comprising oligonucleotide barcode conjugated to a cellular dye, wherein the oligonucleotide barcode comprises a plurality of nucleotides; and wherein the cellular dye binds to the cellular component;
b) quantifying endogenous cellular components using the oligonucleotide barcode.

14. The method of claim 13, wherein the oligonucleotide barcode comprises a cellular component barcode comprising a plurality of nucleotides, wherein the plurality of nucleotides is unique with respect to the cellular component bound by the cellular dye.

15. The method of claim 13, wherein the oligonucleotide barcode comprises a sample barcode comprising a plurality of nucleotides, wherein the plurality of nucleotides is unique with respect to the one or more samples.

16. The method of claim 13, wherein the oligonucleotide barcode comprises a single-cell barcode comprising a plurality of nucleotides, wherein the plurality of nucleotides is unique with respect to a single-cell.

17. A kit for quantifying endogenous cellular components in one or more samples; the kit comprises:
a) at least one composition comprising an oligonucleotide barcode conjugated to a cellular dye, wherein the oligonucleotide barcode comprises a plurality of nucleotides; and wherein the cellular dye binds to the cellular component; and
b) at least one primer pair, wherein at least one primer is specific to a region upstream of the oligonucleotide barcode.

18. The kit of claim 17, wherein the oligonucleotide barcode comprises a cellular component barcode, a sample barcode, a single-cell barcode, or a combination thereof.

19. The kit of claim 18, wherein the cellular component barcode comprises a plurality of nucleotides that are unique with respect to the cellular component bound by the cellular dye, wherein the sample barcode comprises a plurality of nucleotides that are unique with respect to the one or more samples; and wherein the single-cell barcode comprises a plurality of nucleotides that are unique with respect to a single-cell.

20. The kit of claim 17, wherein the at least one primer pair amplifies the oligonucleotide barcode.
